(19) United States
(12) Patent Application Publication
(10) Pub. No.: US 2023/0172170 A1
(43) Pub. Date: Jun. 8, 2023

(54) NON-HUMAN ANIMALS HAVING A HUMANIZED CXCL13 GENE

(71) Applicant: Regeneron Pharmaceuticals, Inc., Tarrytown, NY (US)

(72) Inventors: Davor FRLETA, Forest Hills, NY (US); Naxin TU, Pleasantville, NY (US); Justin GRINDLEY, Closter, NJ (US)

(21) Appl. No.: 17/920,069

(22) PCT Filed: Apr. 20, 2021
(86) PCT No.: PCT/US2021/028087

§ 371 Date: Oct. 20, 2022

Related U.S. Application Data
(60) Provisional application No. 63/013,148, filed on Apr. 21, 2020.

(51) Int. Cl. A01K 67/027; C07K 14/52; A61K 49/00

(52) U.S. Cl. CPC A01K 67/0278; A61K 49/0008; C07K 14/521; A01K 2207/15; A01K 2217/15; A01K 2217/072; A01K 2227/105; A01K 2267/0331

(57) ABSTRACT

Disclosed herein are rodents (such as, but not limited to, mice and rats) genetically modified to comprise a humanized Cxcl13 gene. The rodents disclosed herein have been shown to support better engraftment and proliferation of human cells such as chronic lymphocytic leukemic cells. Compositions and methods for making such genetically modified rodents, as well as methods of using such genetically modified rodents for testing candidate therapeutic agents (e.g., candidate anti-cancer drugs), are provided.

### CROSS REFERENCE TO RELATED APPLICATION

This application claims the benefit of priority to U.S. Provisional Application No. 63/013,148, filed Apr. 21, 2020, the entire contents of which are incorporated herein by reference.

### INCORPORATION BY REFERENCE OF SEQUENCE LISTING

The Sequence Listing in the ASCII text file, named as 38357WO_10574W001_SequenceListing of 14 KB, created on Apr. 16, 2021 and submitted to the United States Patent and Trademark Office via EFS-Web, is incorporated herein by reference.

### BACKGROUND

Non-human animals including rodents have been used as recipients of human cells such as human hematopoietic stem cells, or patient-derived xenograft tissue or cells. Improved non-human animal systems are desired that support and promote survival and proliferation of the engrafted human cells, which will facilitate a better understanding of pathogenesis of a relevant disease and development of therapeutics.

### SUMMARY

It has been established in accordance with this disclosure that humanization of a rodent endogenous Cxcl13 gene results in rodent animals that support better engraftment and proliferation of human cells such as chronic lymphocytic leukemic cells. Accordingly, disclosed herein are Cxcl13 humanized rodent animals, compositions and methods for making such rodents, as well as methods of using such rodents for testing candidate therapeutic agents (e.g., candidate anti-cancer drugs).

In one aspect, disclosed herein is a genetically modified rodent animal comprising in its genome a humanized Cxcl13 gene comprising a rodent Cxcl13 nucleic acid sequence and a human CXCL 13 nucleic acid sequence, wherein the humanized Cxcl13 gene encodes a humanized Cxcl13 polypeptide.

In some embodiments, the humanized Cxcl13 polypeptide comprises a chemokine IL-8 like domain substantially identical with the chemokine IL-8 like domain of a human CXCL13 protein. In some embodiments, the humanized Cxcl13 polypeptide comprises a mature protein sequence substantially identical with the mature protein sequence of the human CXCL13 protein. In some embodiments, the human CXCL13 protein comprises the amino acid sequence of SEQ ID NO: 2.

In some embodiments, the humanized Cxcl13 polypeptide comprises a rodent signal peptide. In some such embodiments, the rodent signal peptide is the signal peptide of the endogenous rodent Cxcl13 protein.

In some embodiments, the human CXCL13 nucleic acid sequence in a humanized Cxcl13 gene encodes a polypeptide substantially identical with the chemokine IL-8 like domain of a human CXCL 13 protein. In some embodiments, the human CXCL 13 nucleic acid sequence comprises exons 3-4 of a human CXCL13 gene. In some embodiments, the human CXCL13 nucleic acid sequence encodes a polypeptide substantially identical with the mature protein sequence of a human CXCL13 protein. In some embodiments, the human CXCL13 nucleic acid sequence comprises exon 3, exon 4 and the coding portion of exon 5 of a human CXCL13 gene. In some embodiments, the human CXCL13 nucleic acid sequence comprises exon 3, exon 4 and exon 5 of a human CXCL13 gene.

In some embodiments, the rodent Cxcl13 nucleic acid sequence in a humanized Cxcl13 gene comprises exon 1 of a rodent Cxcl13 gene. In some embodiments, the rodent Cxcl13 gene is an endogenous Cxcl13 gene.

In some embodiments, the humanized Cxcl13 gene comprises exon 1 of a rodent Cxcl13 gene and exons 3-5 of a human CXCL13 gene.

In some embodiments, the humanized Cxcl13 gene is operably linked to a rodent Cxcl13 promoter. In some embodiments, the rodent Cxcl13 promoter is the endogenous rodent Cxcl13 promoter at an endogenous rodent Cxcl13 locus. In other embodiments, the humanized Cxcl13 gene is operably linked to a human Cxcl13 promoter, optionally at the endogenous rodent Cxcl13 locus.

In some embodiments, the humanized Cxcl13 gene is located at a locus other than an endogenous rodent Cxcl13 locus. In some embodiments, the humanized Cxcl13 gene is located at an endogenous rodent Cxcl13 locus; and in some such embodiments, the humanized Cxcl13 gene may be formed as a result of replacement of a rodent Cxcl13 genomic DNA at an endogenous rodent Cxcl13 locus with a human CXCL 13 nucleic acid. In some embodiments, the humanized Cxcl13 gene is formed as a result of replacement of a rodent genomic DNA comprising exons 2-3 and the coding portion of exon 4 of the rodent Cxcl13 gene at the endogenous rodent Cxcl13 locus with exons 3-5 of a human CXCL13 gene.

In some embodiments, the rodent animals disclosed herein are heterozygous for a humanized Cxcl13 gene. In some embodiments, the rodent animals disclosed herein are homozygous for a humanized Cxcl13 gene.

In some embodiments, the genome of rodents disclosed herein may further comprise a humanized Sirpα gene at an endogenous rodent Sirpα locus, a humanized Baff gene at an endogenous rodent Baff locus, a humanized April gene at an endogenous rodent April locus, a humanized IL-6 gene at an endogenous rodent IL-6 locus, or a combination thereof.

In some embodiments, the rodent animals disclosed herein are immunodeficient, such as a RAG2 and IL-2RG double knockout rodent.

In some embodiments, the rodent animals disclosed herein are selected from a mouse or a rat.

In another aspect, disclosed herein is an isolated rodent tissue or cell, whose genome comprises a humanized Cxcl13 gene described herein. In some embodiments, the isolated rodent cell is a rodent embryonic stem cell. A rodent embryo comprising a rodent embryonic stem cell is also disclosed.

In a further aspect, disclosed herein is a method of making a genetically modified rodent, comprising modifying a rodent genome to comprise a humanized Cxcl13 gene, wherein the humanized Cxcl13 gene comprises a rodent Cxcl13 nucleic acid sequence and a human CXCL13 nucleic acid sequence, and encodes a humanized Cxcl13 polypeptide comprising a chemokine IL-8 like domain substantially identical with the chemokine IL-8 like domain of a human CXCL13 protein; and making a rodent comprising the modified rodent genome.

In some embodiments, a rodent genome is modified by introducing a nucleic acid molecule comprising a human CXCL13 nucleic acid sequence into the genome of a rodent embryonic stem (ES) cell, obtaining a rodent ES cell in which the human CXCL 13 nucleic acid sequence has been integrated into an endogenous rodent Cxcl13 locus to replace a rodent Cxcl13 genomic DNA at the endogenous rodent Cxcl13 locus, thereby forming a humanized Cxcl13 gene; and generating a rodent animal using the obtained rodent ES cell.

In some embodiments, the human CXCL13 nucleic acid sequence being introduced into a rodent ES cell encodes a polypeptide substantially identical with the chemokine IL-8 like domain of the human CXCL13 protein. In some embodiments, the human CXCL13 nucleic acid sequence comprises exons 3-4 of a human CXCL13 gene. In some embodiments, the human CXCL13 nucleic acid sequence encodes a polypeptide substantially identical with the mature protein sequence of the human CXCL13 protein. In some embodiments, the human CXCL13 nucleic acid sequence comprises exon 3, exon 4 and the coding portion of exon 5 of a human CXCL13 gene. In some embodiments, the human CXCL13 nucleic acid sequence comprises exons 3-5 of a human CXCL13 gene.

In some embodiments, the humanized Cxcl13 gene formed at an endogenous rodent Cxcl13 locus comprises exon 1 of the endogenous rodent Cxcl13 gene and exons 3-5 of a human CXCL13 gene, operably linked to the endogenous rodent Cxcl13 promoter at the endogenous rodent Cxcl13 locus.

In another aspect, a targeting nucleic acid construct is disclosed useful for making a genetically modified animal. The targeting construct may comprise a human CXCL13 nucleic acid sequence to be integrated into a rodent Cxcl13 gene at an endogenous rodent Cxcl13 locus, flanked by a 5′ nucleotide sequence and a 3′ nucleotide sequence that are homologous to nucleotide sequences at the endogenous rodent Cxcl13 locus, wherein integration of the human CXCL13 nucleic acid sequence into the endogenous rodent Cxcl13 gene results in a replacement of a rodent Cxcl13 genomic DNA with the human CXCL13 nucleic acid sequence thereby forming a humanized Cxcl13 gene. In some embodiments, the human CXCL13 nucleic acid sequence on the targeting construct comprises exons 3-4 of a human CXCL13 gene. In some embodiments, the human CXCL13 nucleic acid sequence on the targeting construct comprises exon 3, exon 4 and the coding portion of exon 5 of a human CXCL13 gene. In some embodiments, the human CXCL13 nucleic acid sequence on the targeting construct comprises exons 3-5 of a human CXCL13 gene.

In still another aspect, a method is disclosed for testing a candidate agent for treating a disease. The method comprises introducing cells derived from a human subject suffering the disease into a genetically modified rodent animal disclosed herein, contacting the rodent animal with a candidate agent, and assaying if candidate agent is efficacious in reducing or eliminating the cells. A candidate agent effective in reducing or eliminating the cells from the rodent is considered useful for treating the disease.

In some embodiments, the disease is cancer, e.g., a solid tumor or a blood cancer. In some embodiments, the disease is a leukemia.

In some embodiments, a candidate agent is an anti-cancer compound, optionally selected from a small molecule compound, a nucleic acid molecule (e.g., siRNA), or an antibody.

### BRIEF DESCRIPTION OF THE DRAWINGS

The accompanying drawings, which are incorporated in and constitute a part of this specification, illustrate several embodiments and together with the description illustrate the disclosed compositions and methods.

Unless specifically indicated, the figures use empty boxes for human exon sequences, closed boxes for mouse exon sequences, single line for mouse introns and double line for human introns, empty boxes with text therein (e.g., Loxp) for the selection cassette.

FIG. 1A depicts an exemplary strategy for humanization of a mouse Cxcl13 locus. A contiguous mouse Cxcl13 genomic fragment at an endogenous mouse Cxcl13 locus, which comprises mouse exons 2-3 (which encode mouse Cxcl13 chemokine domains) and the coding portion of mouse exon 4 is replaced by a human CXCL13 genomic fragment which comprises human CXCL13 exons 3-4 (encoding human CXCL13 chemokine domains) and human CXCL13 exon 5 (including the 3′ UTR of human CXCL13 as part of exon 5). The replacement results in a humanized Cxcl13 gene at the endogenous mouse Cxcl13 locus, which comprises the mouse native Cxcl13 promoter, in operable linkage to mouse Cxcl13 exon 1 and human CXCL13 exons 3-5 (including human CXCL13 3′ UTR), followed by the 3′ UTR of mouse Cxcl13. The human CXCL13 genomic fragment used in the humanization can include a selection cassette (such as a hygromycin resistance gene under control of a ubiquitin promoter, flanked by LoxP sites) to facilitate screening of correctly targeted clone.

FIGS. 1B-1C. FIG. 1B depicts a nucleic acid construct (“BHR donor”) generated and used to modify a mouse Cxcl13 bacterial artificial chromosome (BAC) via bacterial homologous recombination. FIG. 1C depicts the resulting modified BAC, comprising a chimeric Cxcl13 gene.

FIGS. 1D-1E depict mouse genome heterozygous for a humanized Cxcl13 locus with a selection cassette (1D) and after the cassette has been deleted (1E). The names and locations of the primers and probes used in a modification of allele (MOA) assay (described in Example 1) are also indicated.

FIG. 1F shows alignment of mouse Cxcl13 (SEQ ID NO: 4), human CXCL13 (SEQ ID NO: 2), and humanized (hybrid) Cxcl13 (SEQ ID NO: 8) protein sequences. The signal peptide and the chemokine IL-8-like domain of the proteins are boxed. The two N-terminal cysteines within the chemokine IL-8-like domains are indicated by the arrows. The triangles represent where introns would be (position and size, mouse at the top and human at the bottom), illustrating that the chemokine IL-8-like domain is encoded by the second and third coding exons (exons 2-3 of mouse Cxcl13 or exons 3-4 of human CXCL13).

FIG. 2 shows that mice heterozygous for Cxcl13 humanization as described in Example 1 expressed mature human CXCL13 protein in serum (right), with mice without Cxcl13 humanization as control (left). Mice generated from two positively targeted ES cell clones (clone 1 and clone 2) were examined. Each dot represents one mouse. All mice were from non-engrafted SIRPα^{hu/hu} RAG2^{-/-} IL2Rγ^{-/-} background.

FIG. 3 depicts the results of comparing Cxcl13 humanized mice to NSG mice as hosts for CLL PDX. Each line represents one engrated CLL patient sample. The average of 3-4 engrafted mice per CLL sample was shown. SRG-BA6-13 mice (heterozygous for Cxcl13 humanization) enhanced CLL cell engraftment and proliferation relative to NSG mice, while SRG-BA6 mice (without Cxcl13 humanization) did not.

### DETAILED DESCRIPTION

Disclosed herein are rodents (such as, but not limited to, mice and rats) genetically modified to comprise a humanized Cxcl13 gene. The rodents disclosed herein have been shown to support better engraftment and proliferation of human cells such as chronic lymphocytic leukemic cells. Compositions and methods for making such genetically modified rodents, as well as methods of using such genetically modified rodents for testing candidate therapeutic agents (e.g., candidate anti-cancer drugs), are provided and further described below.

### CXCL13 Gene

C-X-C motif chemokine ligand 13 (CXCL13), also known as B lymphocyte chemoattractant (BLC) or B cell-attracting chemokine 1 (BCA-1), is a protein ligand belonging to the CXC chemokine family. The two N-terminal cysteines of CXC chemokines are separated by one amino acid, represented in this name with an “X”.

CXCL13 is selectively chemotactic for B cells and follicular B helper T cells (or T_{FH} cells), and elicits its effects by interacting with chemokine receptor CXCR5. CXCL13 is strongly expressed in the liver, spleen, lymph nodes, and Peyer’s patches and is believed to be a critical chemokine for attracting B cells and T_{FH} cells to the germinal center for B cell activation, class switching, and somatic hyper-mutation.

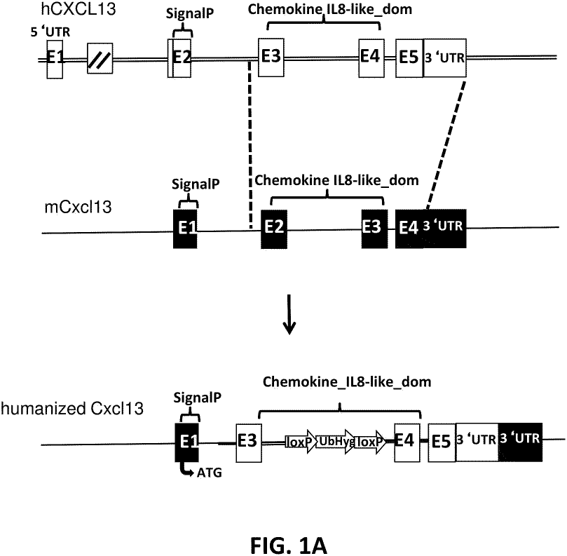

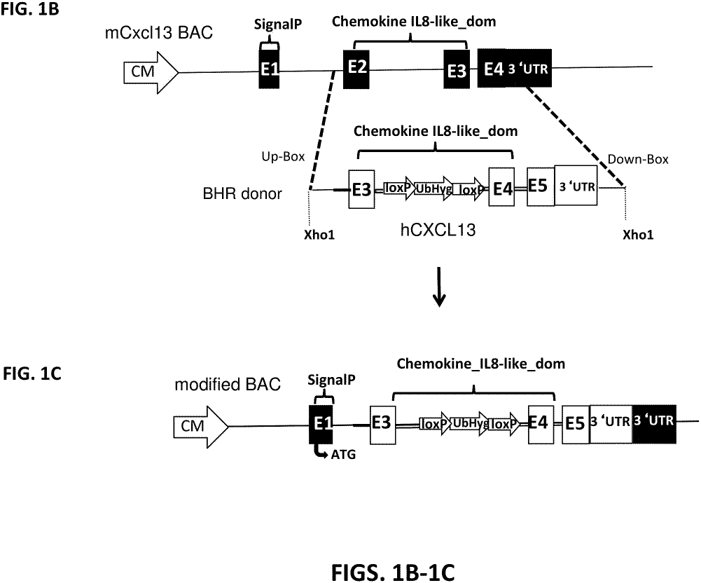

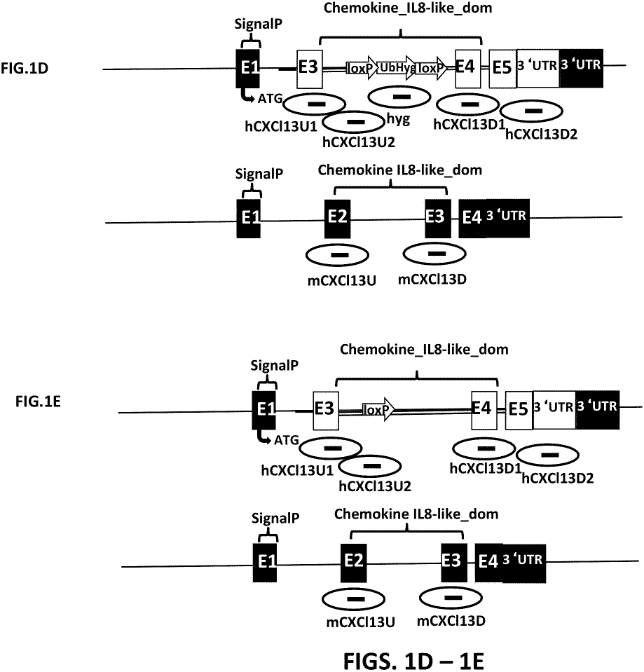

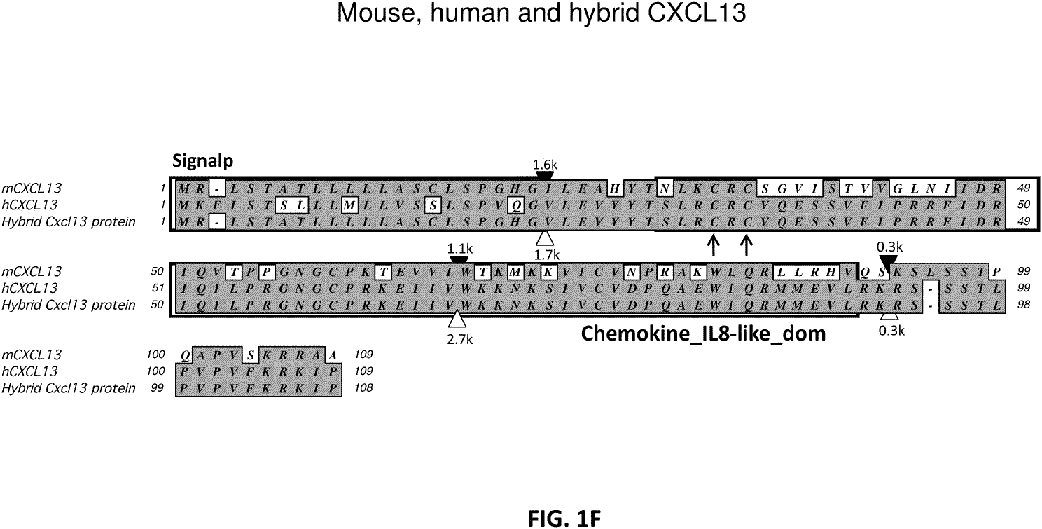

Exemplary sequences, including human CXCL13, mouse Cxcl13, and rat Cxcl13 nucleic acid and protein sequences, as well as exemplary humanized Cxcl13 nucleic acid and protein sequences, are disclosed in the Sequence Listing and summarized in Table 1. An alignment of human CXCL13, mouse Cxcl13, and humanized (hybrid) Cxcl13 protein sequences is provided in FIG. 1F.

TABLE 1

| SEQ ID NO | Description | Features |
|---|---|---|
| 1 | Homo sapiens CXCL13 mRNA, NM_006419.2 | Length: 1219 bp CDS: 79-408 Signal peptide: 79-144 Mature protein: 145-405 5 Exons with exon 2 being the first coding exon: 1...36, 37...142, 143...275, 276...356, 357...1203 |
| 2 | Homo sapiens CXCL13 protein, NP_006410.1 | Length: 109 aa Signal peptide: aa 1-22 Mature: aa 23-109 Chemokine IL-8-like domain: aa 30-91 |
| 3 | Mus musculus Cxcl13 mRNA, NM_018866.2 | Length: 1162 bp CDS: 33-362 Signal peptide: 33-95 Mature protein: 96-359 4 Exons with exon 1 being the first coding exon: 1...93, 94...226, 227...307, 308...1162 |
| 4 | Mus musculus Cxcl13 Protein, NP_061354.1 | Length: 109 aa Signal peptide: 1-21 Mature protein: 22-109 Chemokine IL-8-like domain: aa 29-90 |
| 5 | Rattus norvegicus Cxcl13 mRNA, NM_001017496.1 | Length: 1138 bp CDS: 23-349 Signal peptide: 23-85 Mature protein: 86-349 4 Exons with exon 1 being the first coding exon: 1...83, 84...216, 217...297, 298...1089 |
| 6 | Rattus norvegicus Cxcl13 Protein, NP_001017496.1 | Length: 108 aa Signal peptide: 1-21 Mature protein: 22-108 |
| 7 | Humanized mouse/human chimeric Cxcl13 CDS | Length: 327 bp |
| 8 | Humanized mouse/human chimeric Cxcl13 Protein | Length: 108 aa Signal peptide: 1-21 (from mouse) Mature protein: 22-108 (from human) Chemokine IL-8-like domain: aa 29-90 |

### Cxc113 Humanized Rodents

The rodents disclosed herein comprise a humanized Cxcl13 gene in the germline.

In some embodiments, a rodent disclosed herein comprises a humanized Cxcl13 gene in its genome that includes a nucleotide sequence of a rodent Cxcl13 gene and a nucleotide sequence of a human CXCL13 gene. As used herein, “a nucleotide sequence of a gene” includes a genomic sequence, an mRNA or cDNA sequence, in full or in part of the gene. For example, a nucleotide sequence of a human CXCL13 gene can be a genomic sequence, an mRNA sequence, or a cDNA sequence, in full or in part of the human CXCL13 gene; and a nucleotide sequence of a rodent Cxcl13 gene can be a genomic sequence, an mRNA sequence, or a cDNA sequence, in full or in part of the rodent Cxcl13 gene (e.g., the endogenous rodent Cxcl13 gene). The nucleotide sequence of the rodent Cxcl13 gene and the nucleotide sequence of the human CXCL13 gene are operably linked to each other such that the humanized Cxcl13 gene in the rodent genome encodes a humanized Cxcl13 protein that shares the conserved protein structure with human CXCL13 and rodent Cxcl13 proteins, i.e., being composed of a signal peptide and a mature protein comprising a chemokine IL-8-like domain having an N-terminal C-X-C motif.

In some embodiments, a genetically modified rodent comprises a humanized Cxcl13 gene in its genome, wherein the humanized Cxcl13 gene encodes a humanized Cxcl13 protein that comprises a chemokine IL-8-like domain that is substantially identical with the chemokine IL-8-like domain of a human CXCL13 protein.

In some embodiments, a chemokine IL-8-like domain that is substantially identical with the chemokine IL-8-like domain of a human CXCL13 protein is a polypeptide that is at least 95%, at least 98%, at least 99% or 100% identical with the chemokine IL-8-like domain of a human CXCL13 protein. In some embodiments, a chemokine IL-8-like domain that is substantially identical with the chemokine IL-8-like domain of a human CXCL13 protein is a polypeptide that differs from the chemokine IL-8-like domain of a human CXCL13 protein by not more than 3, 2 or 1 amino acid(s). In some embodiments, a chemokine IL-8-like domain that is substantially identical with the chemokine IL-8-like domain of a human CXCL13 protein is a polypeptide that differs from the chemokine IL-8-like domain of a human CXCL13 protein only at the N— or C— terminal portion of the domain, e.g., by having addition, deletion or substitution of amino acids, but not more than 3, 2 or 1 amino acid(s) at the N— or C— terminal portion of the domain. By “the N— or C-terminal portion of the domain” is meant within 5 amino acids from the N— or C— terminus of the domain.

In some embodiments, the chemokine IL-8-like domain of a human CXCL13 protein comprises amino acids 30-91 of SEQ ID NO: 2. Accordingly, in some embodiments, a genetically modified rodent comprises a humanized Cxcl13 gene in its genome that encodes a humanized Cxcl13 protein that comprises a chemokine IL-8-like domain that is substantially identical with the amino acid sequence as set forth in amino acids 30-91 of SEQ ID NO: 2. In specific embodiments, a genetically modified rodent comprises a humanized Cxcl13 gene in its genome that encodes a humanized Cxcl13 protein that comprises a chemokine IL-8-like domain whose amino acid sequence is set forth in amino acids 30-91 of SEQ ID NO: 2.

In some embodiments, a genetically modified rodent comprises a humanized Cxcl13 gene in its genome, wherein the humanized Cxcl13 gene encodes a humanized Cxcl13 protein that comprises a mature protein sequence that is substantially identical with the mature protein sequence of a human CXCL13 protein.

In some embodiments, a mature protein sequence that is substantially identical with the mature protein sequence of a human CXCL13 protein is a polypeptide sequence that is at least 95%, at least 98%, at least 99% or 100% identical with the mature protein of a human CXCL13 protein. In some embodiments, a mature protein sequence that is substantially identical with the mature protein sequence of a human CXCL13 protein is a polypeptide sequence that differs from the mature protein sequence of a human CXCL13 protein by not more than 3, 2 or 1 amino acid(s). In some embodiments, a mature protein sequence that is substantially identical with the mature protein sequence of a human CXCL13 protein is a polypeptide that differs from the mature protein sequence of a human CXCL13 protein only at the N— or C— terminal portion of the domain, e.g., by having addition, deletion or substitution of amino acids, but not more than 3, 2 or 1 amino acid(s) at the N— or C— terminal portion of the mature protein. By “the N— or C— terminal portion of the mature protein” is meant within 5 amino acids from the N— or C— terminus of the mature protein.

In some embodiments, the mature protein sequence of a human CXCL13 protein comprises amino acids 23-109 of SEQ ID NO: 2. Accordingly, in some embodiments, a genetically modified rodent comprises a humanized Cxcl13 gene in its genome that encodes a humanized Cxcl13 protein that comprises a mature protein sequence that is substantially identical with the amino acid sequence as set forth in amino acids 23-109 of SEQ ID NO: 2. In specific embodiments, a genetically modified rodent comprises a humanized Cxcl13 gene in its genome that encodes a humanized Cxcl13 protein that comprises a mature protein sequence as set forth in amino acids 23-109 of SEQ ID NO: 2.

In some embodiments, the humanized Cxcl13 gene encodes a humanized Cxcl13 protein that comprises a signal peptide that is substantially identical with the signal peptide of a human CXCL13 protein. For example, a humanized Cxcl13 protein may comprise a signal peptide that is at least 95%, at least 98%, at least 99% or 100% identical in sequence with the signal peptide of a human CXCL13 protein, or that differs from the signal peptide of a human CXCL13 protein by not more than 3, 2 or 1 amino acid(s). In specific embodiments, the signal peptide of a human CXCL13 protein comprises the amino acid sequence as set forth in amino acids 1-22 of SEQ ID NO: 2.

In some embodiments, the humanized Cxcl13 gene encodes a humanized Cxcl13 protein that comprises a signal peptide that is substantially identical with the signal peptide of a rodent Cxcl13 protein, such as an endogenous rodent Cxcl13 protein. For example, a humanized Cxcl13 protein may comprises a signal peptide that is at least 95%, at least 98%, at least 99% or 100% identical in sequence with the signal peptide of a rodent Cxcl13 protein, or that differs from the signal peptide of a rodent Cxcl13 protein protein by not more than 3, 2 or 1 amino acid(s). In specific embodiments, the signal peptide of a mouse Cxcl13 protein comprises the amino acid sequence as set forth in amino acids 1-21 of SEQ ID NO: 4. In other specific embodiments, the signal peptide of a rat Cxcl13 protein comprises the amino acid sequence as set forth in amino acids 1-21 of SEQ ID NO: 6.

As described above, the humanized Cxcl13 gene in the genome of a genetically modified rodent includes a nucleotide sequence of a human CXCL13 gene (or “a human CXCL13 nucleotide sequence”) and a nucleotide sequence of an endogenous rodent Cxcl13 gene (or “an endogenous rodent Cxcl13 nucleotide sequence”).

In some embodiments, the human CXCL13 nucleotide sequence encodes a polypeptide substantially identical to the chemokine IL-8 like domain of the human CXCL13 protein encoded by the human CXCL13 gene (e.g., a polypeptide that is at least 95%, at least 98%, at least 99% or 100% identical with the chemokine IL-8-like domain of a human CXCL13 protein; a polypeptide that differs from the chemokine IL-8-like domain of a human CXCL13 protein by not more than 3, 2 or 1 amino acid(s); or a polypeptide that differs from the chemokine IL-8-like domain of a human CXCL13 protein only at the N— or C- terminal portion of the domain, e.g., by having addition, deletion or substitution of amino acids, but not more than 3, 2 or 1 amino acid(s) at the N— or C— terminal portion of the domain). In some embodiments, the human CXCL13 nucleotide sequence is a cDNA sequence. In some embodiments, the human CXCL13 nucleotide sequence is a genomic fragment of a human CXCL13 gene. In some embodiments, the human CXCL13 nucleotide sequence is a genomic fragment comprising exons 3-4 of a human CXCL13 gene.

In some embodiments, the human CXCL13 nucleotide sequence encodes a polypeptide substantially identical to the mature protein sequence of the human CXCL13 protein encoded by the human CXCL13 gene (e.g., a polypeptide that is at least 95%, at least 98%, at least 99% or 100% identical with the mature protein sequence of a human CXCL13 protein; a polypeptide that differs from the mature protein sequence of a human CXCL13 protein by not more than 3, 2 or 1 amino acid(s); or a polypeptide that differs from the mature protein sequence of a human CXCL13 protein only at the N— or C— terminal portion of the domain, e.g., by having addition, deletion or substitution of amino acids, but not more than 3, 2 or 1 amino acid(s) at the N— or C— terminal portion of the domain). In some embodiments, the human CXCL13 nucleotide sequence is a cDNA sequence. In some embodiments, the human CXCL13 nucleotide sequence is a genomic fragment of a human CXCL13 gene. In some embodiments, the human CXCL13 nucleotide sequence is a genomic fragment comprising exon 3, exon 4 and the coding portion of exon 5 (being the last coding exon) of a human CXCL13 gene.

In some embodiments, the human CXCL13 nucleotide sequence is a genomic fragment comprising exon 3 through exon 5 of a human CXCL13 gene, including the 3′ UTR of exon 5. In some such embodiments, the human CXCL13 nucleotide sequence further comprises a 3′ portion of intron 2 of the human CXCL13 gene.

In some embodiments, the rodent Cxcl13 nucleotide sequence in a humanized Cxcl13 gene encodes a polypeptide substantially identical to the signal peptide of a rodent Cxcl13 protein (e.g., a polypeptide that is at least 95%, at least 98%, at least 99% or 100% identical in sequence with the signal peptide of a rodent Cxcl13 protein, or that differs from the signal peptide of a rodent Cxcl13 protein protein by not more than 3, 2 or 1 amino acid(s)). In some embodiments, the rodent Cxcl13 nucleotide sequence comprises exon 1 of a rodent Cxcl13 gene. In specific embodiments, the rodent Cxcl13 nucleotide sequence comprises exon 1 of an endogenous rodent Cxcl13 gene; and in some such embodiments, the rodent Cxcl13 nucleotide sequence includes exon 1 and a 5′ portion of intron 1 of the endogenous rodent Cxcl13 gene.

In some embodiments, the humanized Cxcl13 gene is operably linked to rodent Cxcl13 regulatory sequences, e.g., 5′ transcriptional regulatory sequence(s) such as promoter and/or enhancer, such as endogenous rodent 5′ transcriptional regulatory sequence(s) at an endogenous Cxcl13 locus, such that expression of the humanized Cxcl13 gene is under control of the rodent Cxcl13 5′ regulatory sequence(s).

In some embodiments, the humanized Cxcl13 gene is at an endogenous rodent Cxcl13 locus. In some embodiments, the humanized Cxcl13 gene is at a locus other than an endogenous rodent Cxcl13 locus; e.g., as a result of random integration. In some embodiments where the humanized Cxcl13 gene is at a locus other than an endogenous rodent Cxcl13 locus, the rodents are incapable of expressing a rodent Cxcl13 protein, e.g., as a result of inactivation (e.g., deletion in full or in part) of the endogenous rodent Cxcl13 gene.

In some embodiments where a humanized Cxcl13 gene is at an endogenous rodent Cxcl13 locus, the humanized Cxcl13 gene may result from a replacement of a nucleotide sequence of an endogenous rodent Cxcl13 gene at the endogenous rodent Cxcl13 locus with a nucleotide sequence of a human CXCL13 gene.

In some embodiments, the nucleotide sequence of an endogenous rodent Cxcl13 gene at an endogenous rodent Cxcl13 locus that is being replaced is a genomic fragment of an endogenous rodent Cxcl13 gene that encodes substantially the chemokine IL-8-like domain of the rodent Cxcl13 protein. In some embodiments, a rodent genomic fragment being replaced comprises exons 2-3 of an endogenous rodent Cxcl13 gene.

In some embodiments, the nucleotide sequence of a human CXCL13 gene that replaces a genomic fragment of a rodent Cxcl13 gene at an endogenous rodent Cxcl13 locus is a cDNA sequence. In some embodiments, the human CXCL13 nucleotide sequence that replaces a genomic fragment of a rodent Cxcl13 gene at an endogenous rodent Cxcl13 locus is a genomic fragment of a human CXCL13 gene. In some embodiments, a genomic fragment of a human CXCL13 gene that replaces a genomic fragment of a rodent Cxcl13 gene at an endogenous rodent Cxcl13 locus includes exons, in full or in part, of a human CXCL13 gene, that encode substantially the chemokine IL-8-like domain of the human CXCL13 protein (i.e., that encode a polypeptide substantially identical with the chemokine IL-8-like domain of the human CXCL13 protein). In some embodiments, a genomic fragment of a human CXCL13 gene that replaces a genomic fragment of a rodent Cxcl13 gene at an endogenous rodent Cxcl13 locus includes exons, in full or in part, of a human CXCL13 gene, that encode substantially the mature protein sequence of the human CXCL13 protein (i.e., that encode a polypeptide substantially identical with the mature protein sequence of the human CXCL13 protein). In some embodiments, the human genomic fragment comprises exons 3-4 of a human CXCL13 gene. In some embodiments, the human genomic fragment comprises exons 3-4 and the coding portion of exon 5 of a human CXCL13 gene. In some embodiments, the human genomic fragment comprises exon 3 through exon 5 of a human CXCL13 gene (i.e., including the 3′ UTR of exon 5).

In some embodiments, the genomic sequence of an endogenous rodent Cxcl13 gene that remains at an endogenous rodent Cxcl13 locus after the replacement and is operably linked to the inserted human CXCL13 nucleotide sequence encodes substantially the signal peptide of the endogenous rodent Cxcl13 protein. In some embodiments, the genomic sequence of an endogenous rodent Cxcl13 gene that remains at an endogenous rodent Cxcl13 locus after the replacement includes exon 1 of the endogenous rodent Cxcl13 gene.

In some embodiments, a genomic fragment comprising exons 2-3 and the coding portion of exon 4 of an endogenous rodent Cxcl13 gene at an endogenous rodent Cxcl13 locus has been replaced with a genomic fragment comprising exons 3-5 of a human CXCL13 gene. As a result, a humanized Cxcl13 gene is formed at the endogenous rodent Cxcl13 locus and comprises exon 1 of the endogenous rodent Cxcl13 gene and exons 3-5 of the human CXCL13 gene (including the 3′ UTR of human exon 5), followed by the 3′ UTR of the endogenous rodent Cxcl13 gene. Such humanized Cxcl13 gene encodes a humanized Cxcl13 protein that comprises an endogenous rodent signal peptide and a mature human CXCL13 polypeptide.

In some embodiments, a rodent provided herein is heterozygous for a humanized Cxcl13 gene in its genome. In some embodiments, a rodent provided herein is homozygous for a humanized Cxcl13 gene in its genome.

In some embodiments, a humanized Cxcl13 gene results in an expression of the encoded humanized Cxcl13 protein in a rodent, e.g., in the serum of the rodent. In some embodiments, a humanized Cxcl13 protein is expressed in a pattern similar to, or substantially the same as, a counterpart rodent Cxcl13 protein in a control rodent (e.g., a rodent without the humanized Cxcl13 gene but comprising the fully rodent endogenous Cxcl13 gene); e.g., expression in the liver, spleen, lymph nodes, and Peyer’s patches of the rodent. In some embodiments, a humanized Cxcl13 protein is expressed at a level comparable with, or substantially the same as, a counterpart rodent Cxcl13 protein in a control rodent (e.g., a rodent without the humanized Cxcl13 gene but comprising the fully rodent endogenous Cxcl13 gene); e.g., not differing in its expression by more than 50%, 75%, or 100%.

In some embodiments, rodents disclosed herein are incapable of expressing a rodent Cxcl13 protein, e.g., as a result of inactivation (e.g., deletion in full or in part) or replacement (in full or in part) of the endogenous rodent Cxcl13 gene.

### Additional Optional Genetic Features in Cxc113 Humanized Rodents

In some embodiments, rodents disclosed herein further comprise in their genome a humanized Sirpa gene, a humanized Baff gene, a humanized April gene, a humanized IL-6 gene, or a combination thereof. Humanization of endogenous rodent Sirpα, Baff, April and IL-6 genes is described in WO 2015/042557 A1 (Regeneron Pharmaceuticals Inc.), WO 2015/077071 A1 (Regeneron Pharmaceuticals Inc.), WO 2015/077072 (Regeneron Pharmaceuticals Inc.), and WO 2013/063556 A1 (Regeneron Pharmaceuticals Inc.), respectively, all of which are incorporated herein by reference. In some embodiments, rodents comprise one or more of these additional humanized genes and are homozygous or heterozygous for any of the one or more additional humanized genes. In some embodiments, rodents comprise all of these additional humanized genes and are homozygous or heterozygous for each of these additional humanized genes. In some embodiments, rodents are homozygous or heterozygous for a humanized Cxcl13 gene, comprise one or more or all of these additional humanized genes, and are homozygous or heterozygous for any of these additional humanized genes. In some embodiments, rodents are homozygous for a humanized Cxcl13 gene and homozygous for each of these additional humanized genes.

In some embodiments, rodents disclosed herein further comprise in their genome a humanized Sirpa gene. In some embodiments, the humanized Sirpa gene encodes a humanized Sirpα protein comprising the extracellular domain, in full or in part, of a human SIRPα protein. In some embodiments, the humanized Sirpa gene encodes a humanized Sirpα protein comprising an extracellular portion of a human SIRPα protein responsible for ligand binding (i.e., binding to CD47). In some embodiments, the humanized Sirpa gene encodes a humanized Sirpα protein comprising amino acid residues 28-362 of a human SIRPα protein, e.g., the human SIRPα protein as set forth in GenBank Accession No. NP_001035111.1. In some embodiments, a humanized Sirpα gene comprises exons 2, 3, and 4 of a human SIRPα gene. In some embodiments, a humanized Sirpα gene is located at an endogenous rodent Sirpα locus. In some embodiments, a humanized Sirpα gene is formed as a result of a replacement of exons 2-4 of an endogenous rodent Sirpα gene at an endogenous rodent Sirpα locus by exons 2-4 of a human SIRPα gene. In some embodiments, a humanized Sirpα gene is located at an endogenous rodent Sirpα locus and comprises exon 1 of the endogenous rodent Sirpα gene, exons 2-4 of a human SIRPα gene, and exons 5-8 of the endogenous rodent Sirpα gene, wherein the humanized Sirpα gene is operably linked to the rodent Sirpα promoter at the endogenous rodent Sirpα locus. In some embodiments, rodents disclosed herein are incapable of expressing an endogenous rodent Sirpα protein (e.g., as a result of disruption or replacement of an endogenous rodent Sirpα gene).

In some embodiments, rodents disclosed herein further comprise in their genome a humanized Baff gene. In some embodiments, the humanized Baff gene encodes a humanized Baff protein comprising the extracellular domain, in full or in part, of a human BAFF protein. In some embodiments, the humanized Baff gene encodes a humanized Baff protein comprising an extracellular portion of a human BAFF protein responsible for receptor binding. In some embodiments, the humanized Baff gene encodes a humanized Baff protein comprising amino acid residues 142-285 of a human BAFF protein, e.g., the human BAFF protein as set forth in GenBank Accession No. NP_006564.1. In some embodiments, a humanized Baff gene comprises exons 3-6 of a human BAFF gene. In some embodiments, a humanized Baff gene is located at an endogenous rodent Baff locus. In some embodiments, a humanized Baff gene is formed as a result of a replacement of an endogenous rodent Baff genomic DNA comprising exons 3-6 and the coding portion of exon 7 at an endogenous rodent Baff locus by exons 3-6 of a human BAFF gene. In some embodiments, a humanized Baff gene is located at an endogenous rodent Baff locus and comprises exons 1-2 of the endogenous rodent Baff gene and exons 3-6 of a human BAFF gene, wherein the humanized Baff gene is operably linked to the rodent Baff promoter at the endogenous rodent Baff locus. In some embodiments, rodents disclosed herein are incapable of expressing an endogenous rodent Baff protein (e.g., as a result of disruption or replacement of an endogenous rodent Baff gene).

In some embodiments, rodents disclosed herein further comprise in their genome a humanized April gene. In some embodiments, the humanized April gene encodes a humanized April protein comprising the extracellular domain, in full or in part, of a human APRIL protein. In some embodiments, the humanized April gene encodes a humanized April protein comprising an extracellular portion of a human APRIL protein responsible for receptor binding. In some embodiments, the humanized April gene encodes a humanized April protein comprising amino acid residues 87 to 250 of a human APRIL protein, e.g., the human APRIL protein as set forth in GenBank Accession No. NP_003799.1. In some embodiments, a humanized April gene comprises exons 2-6 of a human APRIL gene. In some embodiments, a humanized April gene is located at an endogenous rodent April locus. In some embodiments, a humanized April gene is formed as a result of a replacement of an endogenous rodent April genomic DNA comprising exon 2 through the coding portion of exon 6 by exons 2-6 of a human APRIL gene. In some embodiments, a humanized April gene is located at an endogenous rodent April locus and comprises exon 1 of the endogenous rodent April gene and exons 2-6 of a human APRIL gene, wherein the humanized April gene is operably linked to the rodent April promoter at the endogenous rodent April locus. In some embodiments, rodents disclosed herein are incapable of expressing an endogenous rodent April protein (e.g., as a result of disruption or replacement of an endogenous rodent April gene).

In some embodiments, rodents disclosed herein further comprise in their genome a humanized IL-6 gene. In some embodiments, the humanized IL-6 gene encodes a human IL-6 protein, e.g., the human IL-6 protein as set forth in GenBank Accession No. NP_000591.1. In some embodiments, a humanized IL-6 gene comprises the coding portion of exon 1 through exon 5 of a human IL-6 gene. In some embodiments, a humanized IL-6 gene is located at an endogenous rodent IL-6 locus. In some embodiments, a humanized IL-6 gene is formed as a result of a replacement of an endogenous rodent IL-6 genomic DNA comprising the coding portion of exon 1 through the coding portion of exon 5 by the coding portion of exon 1 through exon 5 of a human IL-6 gene. In some embodiments, a humanized IL-6 gene is located at an endogenous rodent IL-6 locus, and comprises the noncoding portion of exon 1 of the endogenous rodent IL-6 gene, the coding portion of exon 1 through exon 5 of a human IL-6 gene, wherein the humanized IL-6 gene is operably linked to the rodent IL-6 promoter at the endogenous rodent IL-6 locus. In some embodiments, rodents disclosed herein are incapable of expressing an endogenous rodent IL-6 protein (e.g., as a result of disruption or replacement of an endogenous rodent IL-6 gene).

In some embodiments, rodents disclosed herein further have their RAG2 and IL-2RG genes disupted and are incapable of expressing endogenous RAG2 or IL-2 Receptor gamma chain (also known as “γc”) proteins. RAG2 and IL-2RG double knock-out (DKO) rodents are known immunodeficient rodents (see, e.g., Traggiai E et al. (2004) Development of a human adaptive immune system in cord blood cell-transplanted mice, Science 304:104-107) and readily available commercially (e.g., from Taconic Biosciences, Inc., New York).

In some embodiments, rodents disclosed herein are homozygous for one or more (e.g., all) of a humanized Sirpα gene, a humanized Baff gene, a humanized April gene, all at the respective endogenous locus (e.g., as a result of replacement described above), and are homozygous null for both RAG2 and IL-2RG genes.

In some embodiments, rodents of this disclosure include, as non-limiting examples, a mouse, a rat, and a hamster. In some embodiments, a rodent is selected from the superfamily Muroidea. In some embodiments, a rodent of this disclosure is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, a rodent of this disclosure is selected from a true mouse or rat (family Muridae), a gerbil, a spiny mouse, and a crested rat. In some embodiments, a mouse of this disclosure is from a member of the family Muridae.

In some embodiments, a rodent is a mouse. In some embodiments, the rodent is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, a rodent is a mouse of a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129/SvJae, 129S6 (129/SvEvTac), 129S7, 129S8, 129T1, 129T2 (see, e.g., Festing et al., 1999, Mammalian Genome 10:836; Auerbach et al., 2000, Biotechniques 29(5): 1024-1028, 1030, 1032). In some embodiments, a rodent is a mouse that is a mix of a 129 strain and a C57BL/6 strain. In some embodiments, a rodent is a mouse that is a mix of aforementioned 129 strains, or a mix of aforementioned BL/6 strains. In some embodiments, a rodent is a mouse of a BALB strain, e.g., BALB/c strain. In some embodiments, a rodent is a mouse that is a mix of a BALB strain and another aforementioned strain.

In some embodiments, a rodent is a rat. In some certain embodiments, a rat is selected from a Wistar rat, an LEA strain, a Sprague Dawley strain, a Fischer strain, F344, F6, and Dark Agouti. In some embodiments, a rat strain as described herein is a mix of two or more strains selected from the group consisting of Wistar, LEA, Sprague Dawley, Fischer, F344, F6, and Dark Agouti.

### Tissues and Cells of Genetically Modified Rodents

In some embodiments, disclosed herein is an isolated rodent cell or tissue whose genome comprises a humanized Cxcl13 gene as described herein.

In some embodiments, a tissue is selected from adipose, bladder, brain, breast, bone marrow, eye, heart, intestine, kidney, liver, lung, lymph node, muscle, pancreas, plasma, serum, skin, spleen, stomach, thymus, testis, ovum, and a combination thereof.

In some embodiments, a cell is selected from a dendritic cell, lymphocyte (e.g., a B or T cell), macrophage and a monocyte. In some embodiments, an isolated rodent cell is a rodent embryonic stem cell, or a rodent egg.

### Compositions and Methods for Making Humanized Rodents

Disclosed herein is a targeting vector (or nucleic acid construct) comprising a human CXCL13 nucleotide sequence desired to be integrated into a rodent locus.

In some embodiments, the human CXCL13 nucleotide sequence encodes a polypeptide substantially identical to the chemokine IL-8-like domain of a human CXCL13 protein. In some embodiments, the human CXCL13 nucleotide sequence encodes a polypeptide substantially identical to the amino acid sequence set forth in amino acids 30-91 of SEQ ID NO: 2. In some embodiments, the human CXCL13 nucleotide sequence encodes a polypeptide substantially identical to the mature portion of a human CXCL13 protein. In some embodiments, the human CXCL13 nucleotide sequence encodes a polypeptide substantially identical to the amino acid sequence set forth in amino acids 23-109 of SEQ ID NO: 2.

In some embodiments, the human CXCL13 nucleotide sequence comprises exon 3 and exon 4 of a human CXCL13 gene. In some embodiments, the human CXCL13 nucleotide sequence comprises exon 3, exon 4, and the coding portion of exon 5 of a human CXCL13 gene. In some embodiments, the human CXCL13 nucleotide sequence comprises exon 3 through exon 5 of a human CXCL13 gene.

The targeting vector also includes 5′ and 3′ rodent sequences flanking the human nucleotide sequence to be integrated, also known as 5′ and 3′ homology arms, that mediate homologous recombination and integration of the human nucleotide sequence into the target rodent locus (e.g., an endogenous Cxcl13 locus). Typically, the 5′ and 3′ flanking rodent sequences are the nucleotide sequences that flank the corresponding rodent nucleotide sequence at the target rodent locus that is to be replaced by the human nucleotide sequence. For example, in embodiments where a rodent genomic sequence comprising rodent Cxcl13 exons 2-4 is to be replaced with a human genomic sequence comprising human CXCL13 exons 3-5, the 5′ flanking sequence in the targeting vector can include a 5′ portion of intron 1 of the rodent Cxcl13 gene, and the 3′ flanking sequence can include a 5′ portion of the 3′ UTR in exon 4 of the rodent Cxcl13 gene.

In some embodiments, a targeting vector comprises a selection marker gene. In some embodiments, a targeting vector comprises one or more site-specific recombination sites. In some embodiments, a targeting vector comprises a selection marker gene, flanked by site-specific recombination sites, such that the selection marker gene can be deleted as a result of recombination between the sites.

In an exemplary embodiment, a targeting vector is generated from a bacterial artificial chromosome (BAC) clone carrying a rodent Cxcl13 genomic DNA using bacterial homologous recombination and VELOCIGENE® technology (see, e.g., U.S. 6,586,251 and Valenzuela et al. (2003) Nature Biotech. 21(6):652-659). As a result of bacterial homologous recombination, a rodent genomic sequence is deleted from the BAC clone, and a human nucleotide sequence is inserted, resulting in a modified BAC clone carrying the human nucleotide sequence, flanked with 5′ and 3′ rodent homology arms. In some embodiments, the human nucleotide sequence can be a cDNA sequence or a human genomic DNA encoding the mature portion, or at least the chemokine IL-8-like domain, of the human CXCL13 protein. The modified BAC clone, once linearized, can be introduced into rodent embryonic stem (ES) cells.

In some embodiments, the present invention provides use of a targeting vector as described herein to make a modified rodent embryonic stem (ES) cell. A targeting vector can be introduced into a rodent ES cell by, e.g., electroporation. Both mouse ES cells and rat ES cells have been described in the art. See, e.g., US 7,576,259, US 7,659,442, US 7,294,754, and US 2008-0078000 A1 (all of which are incorporated herein by reference in their entireties) that describe mouse ES cells and the VELOCIMOUSE® method for making a genetically modified mouse; US 2014/0235933 A1 (Regeneron Pharmaceuticals, Inc.), US 2014/0310828 A1 (Regeneron Pharmaceuticals, Inc.), Tong et al. (2010) Nature 467:211-215, and Tong et al. (2011) Nat Protoc. 6(6): doi: 10.1038/nprot.2011.338 (all of which are incorporated herein by reference in their entireties) that describe rat ES cells and methods for making a genetically modified rat, which can be used to make a modified rodent embryo, which in turn can be used to make a rodent animal.

In some embodiments, ES cells already comprising additional desirable genetic feature(s) (e.g., homozygous for a humanized Sirpa gene, a humanized Baff gene, a humanized April gene, humanized IL-6 gene, and/or RAG2-/- and IL-2RG -/-) are used as recipient cells in electroporation with a targeting vector comprising a human CXCL13 nucleotide sequence. In some embodiments, these addittional desirable genetic feature(s) can be introduced later (e.g., by crossing a humanized Cxcl13 rodent with a second rodent having one or more desirable genetic features).

In some embodiments, ES cells having a human CXCL13 nucleotide sequence integrated in the genome can be selected. In some embodiments, ES cells are selected based on loss of rodent allele and/or gain of human allele assays. In some embodiments, selected ES cells are then used as donor ES cells for injection into a pre-morula stage embryo (e.g., 8-cell stage embryo) by using the VELOCIMOUSE® method (see, e.g., US 7,576,259, US 7,659,442, US 7,294,754, and US 2008-0078000 A1, all of which are incorporated by reference in their entireties), or methods described in US 2014/0235933 A1 and US 2014/0310828 A1, which are both incorporated by reference in their entireties. In some embodiments, an embryo comprising the donor ES cells is incubated and implanted into a surrogate mother to produce an F0 rodent. Rodent pups bearing a human nucleotide sequence can be identified by genotyping of DNA isolated from tail snips using loss of rodent allele and/or gain of human allele assays.

In some embodiments, rodents heterozygous for a humanized gene can be crossed to generate homozygous rodents.

A Cxcl13 humanized rodent as described herein can be bred or crossed with another rodent. Accordingly, methods of breeding as well as progenies obtained from such breeding are also embodiments of this disclosure.

In some embodiments, a method is provided which comprises breeding a first rodent as described hereinabove, e.g., a rodent whose genome comprises a humanized Cxcl13 gene, with a second rodent, resulting in a progeny rodent whose genome comprises the humanized Cxcl13 gene. The progeny may possess other desirable phenotypes or genetic modifications inherited from the second rodent used in the breeding. In some embodiments, the progeny rodent is heterozygous for the humanized Cxcl13 gene. In some embodiments, the progeny rodent is homozygous for the humanized Cxcl13 gene.

In some embodiments, a progeny rodent is provided whose genome comprises a humanized Cxcl13 gene, wherein the progeny rodent is produced by a method comprising breeding a first rodent whose genome comprises a humanized Cxcl13 gene, with a second rodent. In some embodiments, the progeny rodent is heterozygous for the humanized Cxcl13 gene. In some embodiments, the progeny rodent is homozygous for the humanized Cxcl13 gene.

In some embodiments, the second rodent comprises in its genome a humanized Sirpα gene, a humanized Baff gene, a humanized April gene, a humanized IL-6 gene, or a combination thereof.

In some embodiments, the second rodent is a RAG2 and IL-2RG double knock-out (DKO) rodent (RAG2-/- and IL-2RG-/-).

In some embodiments, the second rodent comprises in its genome a humanized Sirpα gene, a humanized Baff gene, a humanized April gene, and a humanized IL-6 gene, at their respective endogenous locus, and is RAG2-/- and IL-2RG-/-.

### Methods Employing the Humanized Rodents

Rodents disclosed herein provide a useful in vivo system and source of biological materials for identifying and testing compounds for their potential to treat human diseases.

In some embodiments, rodent animals disclosed herein are used to develop agents that target human CXCL13 and/or modulate CXCL13-CXCR5 interaction. In some embodiments, rodents disclosed herein are used to screen and develop candidate agents (e.g., antibodies) that specifically bind to human CXCL13. In some embodiments, rodent animals disclosed herein are used to determine the binding profile of an agent (e.g., an antihuman CXCL13 antibody).

In some embodiments, rodent animals disclosed herein are used to measure the effect of blocking or modulating human CXCL13 activity. In some embodiments, a rodent animal disclosed herein is exposed to a candidate agent that binds to and inhibits human CXCL13, and is analyzed for effects on human CXCL13-dependent processes. For example, a Cxcl13 humanized rodent animal disclosed herein can be exposed to a candidate agent that binds to and inhibits human CXCL13, and is analyzed for effects of the candidate agent on the development of germinal center, B cell development, and serum human immunoglobulin levels after engraftment of human hemapoietic CD34+ cells. The rodent animal is preferably an immuno deficient animal, e.g., a rodent having a Rag2-/- IL-2RG-/-genotype; and more preferably having a Rag2-/- IL-2RG-/- Sirpα^{hu/hu} Baff^{hu/hu} April^{hu/hu} IL-6^{hu/hu} genotype.

In some embodiments, rodent animals disclosed herein provide an improved in vivo system for maintenance of patient-derived xenograft tissue or cells. In some embodiments, patient-derived xenograft cells are cancer cells. In some embodiments, the cancer cells are cells of a solid tumor. In some embodiments, the cancer cells are cancerous blood cells, e.g., blood cells of patients having leukemia, myeloma, Hodgkin’s or non-Hodgkin’s lymphoma. In some embodiments, patient-derived xenograft cells are chronic lymphocytic leukemic (CCL) cells.

Rodent animals disclosed herein can be employed to assess the efficacy of a therapeutic drug targeting human cancer cells. In various embodiments, a rodent animal disclosed herein is engrafted with human cancer cells (e.g., CCL cells), and a drug candidate targeting such human cancer cells is administered to the rodent animal. The therapeutic efficacy of the drug is then determined by monitoring the human cells in the rodent animal after the administration of the drug, e.g., by assessing whether growth or metastasis of the human cancer cells in the rodent animal is inhibited as a result of the administration of the drug. Drugs that can be tested in the non-human animals include both small molecule compounds, i.e., compounds of molecular weights of less than 1500 kD, 1200 kD, 1000 kD, or 800 dalton, and large molecular compounds (such as proteins, e.g., antibodies or antigen-binding fragments of antibodies), which have intended therapeutic effects for the treatment of human diseases and conditions by targeting (e.g., binding to and/or acting on) human cells.

The present description is further illustrated by the following examples, which should not be construed as limiting in any way. The contents of all cited references (including literature references, issued patents, and published patent applications as cited throughout this application) are hereby expressly incorporated by reference in their entireties.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure.

### Example 1. Generation of Humanized CXCL13 Mouse

The mouse Cxcl13 locus was humanized by using VELOCIGENE® technology (see, e.g., U.S. Pat. No. 6,586,251 and Valenzuela et al. (2003) High-throughput engineering of the mouse genome couple with high-resolution expression analysis. Nat. Biotech. 21(6): 652-659, both incorporated herein by reference). The resulting humanized Cxcl13 locus included the endogenous mouse Cxcl13 promoter, operably linked to mouse Cxcl13 exon 1, human CXCL13 exon 3 and exon 4 (encoding human CXCL13 chemokine domains), and human CXCL13 exon 5 (which includes the human 3′ UTR), followed by the endogenous mouse Cxcl13 3′ UTR. See FIG. 1A.

To humanize the mouse Cxcl13 locus, an initial plasmid was generated to carry a nucleic acid encoding human CXCL13 chemokine domains, flanked by mouse Cxcl13 sequences. More specifically, this initial plasmid contained from 5′ to 3′: (i) aXhoI site, (ii) a mouse Cxcl13 sequence of 249 bp (a 5′ portion of mouse intron 1) (“Up-Box”), (iii) a human CXCL13 nucleic acid sequence which included a 3′ portion of intron 2 (150 bp), exon 3 (133 bp), intron 3 (2778 bp, containing an AgeI site and a SalI site), exon 4 (81 bp), intron 4 (293 bp), and exon 5 (847 bp, including the 3′ UTR) of human CXCL13, (iv) a 5′ portion of the 3′ UTR of mouse Cxcl13 (140 bp, “Down-Box”), and (v) a XhoI site. The plasmid also contained a selection cassette containing a hygromycin resistance gene operably linked to a ubiquitin promoter, flanked by LoxP sites. This BHR donor plasmid (“BHR” for baterial homologous recombination) was then digested with XhoI to release the fragment (referred herein to as the “BHR donor”) containing the human CXCL13 nucleic acid sequence and the selection cassette, flanked by mouse Cxcl13 sequences (the Up-Box and the Down-Box). See FIG. 1B.

The BHR donor was subsequently used to modify a mouse Cxcl13 bacterial artificial chromosome (BAC) clone RP23-162n18 (Thermo-Fisher/Invitrogen). Through bacterial homologous recombination, a contiguous nucleic acid fragment on the mouse Cxcl13 BAC, which included mouse exons 2-3 (encoding mouse Cxcl13 chemokine domains) and the coding portion of mouse exon 4 was replaced by the human CXCL13 nucleic acid sequence from the BHR donor, giving rise to a modified BAC carrying a chimeric, humanized Cxcl13 nucleic acid containing mouse Cxcl13 exon 1, and human CXCL13 exons 3-5 (with the hygromycin cassette inserted between human exons 3 and 4), followed by the 3′ UTR of mouse Cxcl13. See FIGS. 1B-1C.

The modified BAC was then used as a targeting vector and was electroporated into mouse embryonic stem (ES) cells comprising hSIRP-alpha (or human SIRPα), RAG2-/-IL2Rg-/- modifications. Successful integration was confirmed by a modification of allele (MOA) assay as described, e.g., in Valenzuela et al., supra. Primers and probes used for the MOA assay for detecting the presence of human CXCL13 sequences and confirming the loss and/or retention of mouse Cxcl13 sequences are described in Table 2, and their locations are depicted in FIG. 1D. After a correctly targeted ES cell clone was selected, the hygromycin selection cassette was excised by a Cre recombinase. The humanized Cxcl13 locus after the deletion of the cassette is depicted in FIG. 1E. The coding sequence of the resulting humanized Cxcl13 gene and the encoded amino acid sequence are set forth in SEQ ID NO: 7 and SEQ ID NO: 8, respectively. An alignment of human CXCL13 (SEQ ID NO: 2), mouse Cxcl13 (SEQ ID NO: 4) and humanized Cxcl13 (SEQ ID NO: 8) protein sequences is provided in FIG. 1F.

TABLE 2

| Probe Name | Primer/Probe (5′ to 3′) | SEQ ID NO |
|---|---|---|
| hcxcl13U1 | Forward: TTACAGGTGTTCTGGAGGTCTA | 9 |
| | Reverse: CGATCAATGAAGCGTCTAGGGAT | 10 |
| | Probe: ACACAAGCTTGAGGTGTAGATGTGTCCA | 11 |
| hcxcl13U2 | Forward: GCCTGACCAACATGGAGAAAC | 12 |
| | Reverse: GCCTCAGCCTCCCAAGTAG | 13 |
| | Probe: TACAAAATTAGCCGGGTGTGGTGGT | 14 |
| hcxcl13D1 | Forward: CCTCGCAGCTTTGGATTCAAC | 15 |
| | Reverse: GGGCAGTTTGGGATCTGGAAT | 16 |
| | Probe: TGTTCCACAAATGGTTGTTCTCTTATTCC | 17 |
| hcxcl13D2 | Forward: GGCATCAAACTCAGAGATGTGAAG | 18 |
| | Reverse: GACAGGCAAGAGACCAGTCAT | 19 |
| | Probe: TCTAGCCATGACCTACTTGGGAGTAGT | 20 |
| mcxcl13U | Forward: TCGGTTCTACGTCTATGTTCTTTG | 21 |
| | Reverse: GGGCTTCCAGAATACCTGTGAAC | 22 |
| | Probe: TCCAATGGGTTGAAGTGTCTGACTC | 23 |
| mcxcl13D | Forward: TGCACAGCAGCCATCATTTG | 24 |
| | Reverse: CAGGCAGCTCTTCTCTTACTCA | 25 |
| | Probe: TGCCACTAGAGGAAAGCTATGGTTTCC | 26 |
| hyg | Forward: TGCGGCCGATCTTAGCC | 27 |
| | Reverse: TTGACCGATTCCTTGCGG | 28 |
| | Probe: ACGAGCGGGTTCGGCCCATTC | 29 |

Positively targeted ES cells were used as donor ES cells and microinjected into a pre-morula (8-cell) stage mouse embryo by the VELOCIMOUSE® method (see, e.g., US 7,576,259, US 7,659,442, US 7,294,754, and US 2008-0078000 A1, all of which are incorporated herein by reference). The mouse embryo comprising the donor ES cells was incubated in vitro and then implanted into a surrogate mother to produce an F0 mouse fully derived from the donor ES cells. Mice bearing a humanized Cxcl13 gene were identified by genotyping using the MOA assay described above. Mice heterozygous for the humanized Cxcl13 gene were bred to homozygousity.

To determine whether mice homozygous for the Cxcl13 humanization expressed the humanized protein, humanized or control mice (mice expressing mouse CXCL13 protein), all on a non-engrafted hSIRP-alpha Rag2-/- IL-2RG-/- background, were tested. Mice derived from two different clones of humanized CXCL13 ES cells (designated as clone 1 and clone 2) were tested. Mice were euthanized and blood collected via cardiac puncture. Serum was prepared, and human CXCL13 levels in serum assessed by human CXCL13 Quantikine ELISA (R&D systems; Cat # DCX130) according to manufacturer’s instructions.

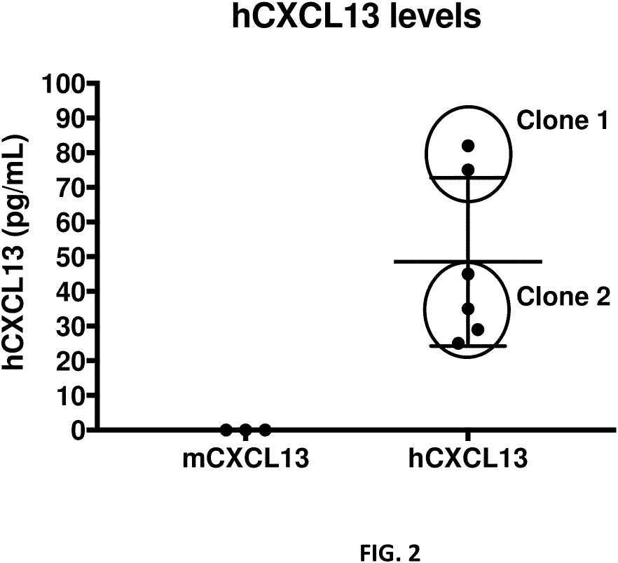

Mice heterozygous for the Cxcl13 humanization as described above were found to express mature human CXCL13 in serum (FIG. 2).

### Example 2. Humanized Cxc113 Mice Exhibited Enhanced Human Chronic Lymphocytic Leukemia Cell Engraftment

### Materials and Methods

Mice - NSG mice were purchased from Jackson Labs. Mice comprising SIRPα^{hu/hu} Rag2^{-/-} IL2Rg^{-/-} and humanized BAFF, APRIL, IL-6 (SRG-BA6), as well as mice comprising SIRPα^{hu/hu} Rag2^{-/-} IL2Rg^{-/-} and humanized BAFF, APRIL, IL-6, and CXCL13 (SRG-BA6-13) were generated at Regeneron, using a combination of successive targeting into ES cells and breeding. Humanization of the mouse Sirpa, Baff, April and IL-6 genes in these mice were described in WO 2015/042557 A1 (Regeneron Pharmaceuticals Inc.), WO 2015/077071 A1 (Regeneron Pharmaceuticals Inc.), WO 2015/077072 (Regeneron Pharmaceuticals Inc.), and WO 2013/063556 A1 (Regeneron Pharmaceuticals Inc.), respectively (all of which are incorporated herein by reference). Mice were between 6-16 week of age and were sub-lethally irradiated (2 Gy) when xenograft was performed.

Patient Materials -- Mononuclear cells from fresh peripheral blood of chronic lymphocytic leukemia (CLL) patients were isolated by gradient centrifugation, then used directly or cryo preserved. Cells were labeled with TraceVilot or CFSC (Invitrogen) according to manufacturer’s protocol and intravenously injected into mice. 5x10⁷ to 1x10⁸ cells were injected into each mouse.

Flow cytometry - Mice were bled retro-orbitally two weeks post xenograft. Red blood cells were lysed. Mononuclear cells were suspended in PBS with 2% fetal bovine serum, stained with antibodies described below, mixed with CountBright absolute counting beads (Invitrogen) and subjected to flow cytometry. The following monoclonal antibodies (mAbs) from Biolegend or eBioscience were used: anti-mCD45 (30F11), anti-Ter119, anti-hCD45 (HI30), anti-hCD3 (UCHT1), anti-hCD19 (HIB19), anti-hCD5 (L17F12). Antibodies were directly coupled to APC, APCCy7, Alexa Fluor 700, BV-605, or BV-711. Data were acquired on a BD Fortessa X20 instrument and analyzed by the Flow Jo program.

Gating strategy - Singlet live cells were gated for hCD45+mCD45- population. CLL cells were further gated as hCD19+hCD5+hCD3-. Proliferating CLL cells were further gated as either TraceViolet-low or CFSE-low.

### Results

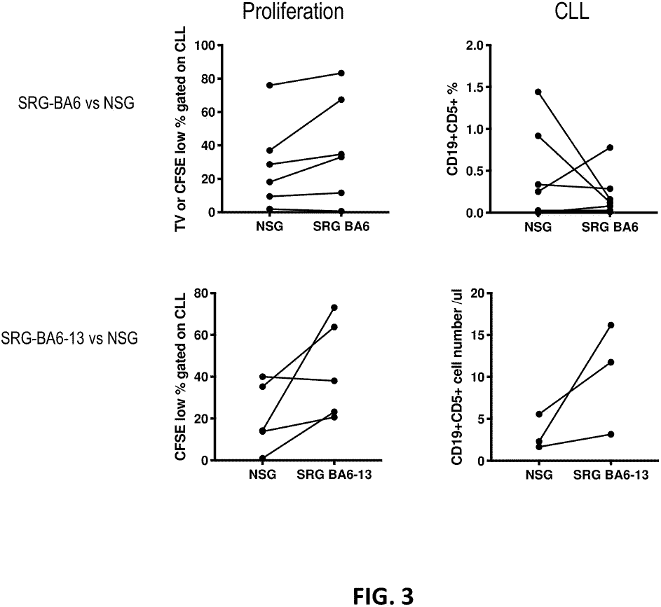

Engraftment of 12 CLL patient samples was compared between NSG and SRG-BA6 mice, or between NSG and SRG-BA6-13 mice. 2-3 mice of each strain were xenografted with the same patient sample. Two weeks post xenograft, mice were bled to assess CLL engraftment. No obvious difference of CLL cell frequency (hCD19+hCD5+hCD3-) and their proliferation status (TraceViolet-low or CFSE-low) was observed between NSG and SRG-BA6 strains. In contrast, proliferation (4 out of 5 patient samples) and CLL cell number (3 out of 3 patient samples) were markedly increased in SRG-BA6-13 mice, as compared to NSG mice (FIG. 3). The results indicate that humanization of the endogenous Cxcl13 gene enhanced the engraftment of CLL patient samples in immunodeficient mice.

What is claimed is:

1. A genetically modified rodent animal comprising in its genome a humanized Cxcl13 gene comprising a rodent Cxcl13 nucleic acid sequence and a human CXCL13 nucleic acid sequence, wherein the humanized Cxcl13 gene encodes a humanized Cxcl13 polypeptide comprising a chemokine IL-8 like domain substantially identical with the chemokine IL-8 like domain of a human CXCL13 protein.

2. The genetically modified rodent animal of claim 1, wherein the humanized Cxcl13 polypeptide comprises a mature protein sequence substantially identical with the mature protein sequence of the human CXCL13 protein.

3. The genetically modified rodent animal of claim 1 or 2, wherein the human CXCL13 protein comprises the amino acid sequence of SEQ ID NO: 2.

4. The genetically modified rodent animal of any one of claims 1-3, wherein the humanized Cxcl13 protein comprises a rodent signal peptide.

5. The genetically modified rodent animal of claim 4, wherein the rodent signal peptide is the signal peptide of the endogenous rodent Cxcl13 protein.

6. The genetically modified rodent animal of claim 1, wherein the human CXCL13 nucleic acid sequence comprises exons 3-4 of a human CXCL13 gene.

7. The genetically modified rodent animal of claim 1, wherein the human CXCL13 nucleic acid sequence comprises exon 3, exon 4 and the coding portion of exon 5 of a human CXCL13 gene.

8. The genetically modified rodent animal of any one of claims 1-7, wherein the human CXCL13 nucleic acid sequence comprises exon 3, exon 4 and exon 5 of a human CXCL13 gene.

9. The genetically modified rodent animal of any one of claims 1-8, wherein the rodent Cxcl13 nucleic acid sequence comprises exon 1 of a rodent Cxcl13 gene.

10. The genetically modified rodent animal of claim 9, wherein the rodent Cxcl13 gene is an endogenous Cxcl13 gene.

11. The genetically modified rodent animal of claim 1, wherein the humanized Cxcl13 gene comprises exon 1 of a rodent Cxcl13 gene and exons 3-5 of a human CXCL13 gene.

12. The genetically modified rodent animal of any one of claims 1-11, wherein the humanized Cxcl13 gene is operably linked to a rodent Cxcl13 promoter.

13. The genetically modified rodent animal of claim 12, wherein the rodent Cxcl13 promoter is the endogenous rodent Cxcl13 promoter at an endogenous rodent Cxcl13 locus.

14. The genetically modified rodent animal of any one of claims 1-12, wherein the humanized Cxcl13 gene is located at an endogenous rodent Cxcl13 locus.

15. The genetically modified rodent animal of claim 14, wherein the humanized Cxcl13 gene is formed as a result of replacement of a rodent Cxcl13 genomic DNA at an endogenous rodent Cxcl13 locus with the human CXCL13 nucleic acid.

16. The genetically modified rodent animal of claim 14, wherein the humanized Cxcl13 gene is formed as a result of replacement of a rodent genomic DNA comprising exons 2-3 and the coding portion of exon 4 of the rodent Cxcl13 gene at the endogenous rodent Cxcl13 locus with exons 3-5 of a human CXCL13 gene.

17. The genetically modified rodent animal of any of the proceeding claims, wherein the rodent is homozygous for the humanized Cxcl13 gene.

18. The genetically modified rodent animal of any of the proceeding claims, whose genome further comprises a humanized Sirpα gene at an endogenous rodent Sirpα locus, a humanized Baff gene at an endogenous rodent Baff locus, a humanized April gene at an endogenous rodent April locus, a humanized IL-6 gene at an endogenous rodent IL-6 locus, or a combination thereof.

19. The genetically modified rodent animal of any of the proceeding claims, wherein the RAG2 gene and the IL-2RG gene have been disrupted.

20. The genetically modified rodent animal of any of the proceeding claims, wherein the rodent is a mouse or a rat.

21. An isolated rodent tissue or cell, whose genome comprises a humanized Cxcl13 gene comprising a rodent Cxcl13 nucleic acid sequence and a human CXCL13 nucleic acid sequence, wherein the humanized Cxcl13 gene encodes a humanized Cxcl13 polypeptide comprising a chemokine IL-8 like domain substantially identical with the chemokine IL-8 like domain of a human CXCL13 protein.

22. The isolated rodent tissue or cell of claim 21, wherein the humanized Cxcl13 gene comprises exon 1 of a rodent Cxcl13 gene and exons 3-5 of a human CXCL13 gene.

23. The isolated rodent tissue or cell of claim 21 or 22, wherein the rodent cell is a rodent embryonic stem cell.

24. The isolated rodent tissue or cell of any one of claims 21-23, wherein the rodent is a mouse or a rat.

25. A rodent embryo, comprising the rodent embryonic stem cell of claim 23.

26. A method of making a genetically modified rodent, comprising:
modifying a rodent genome to comprise a humanized Cxcl13 gene, wherein the humanized Cxcl13 gene comprises a rodent Cxcl13 nucleic acid sequence and a human CXCL13 nucleic acid sequence, and encodes a humanized Cxcl13 polypeptide comprising a chemokine IL-8 like domain substantially identical with the chemokine IL-8 like domain of a human CXCL13 protein; and
making a rodent comprising the modified rodent genome.

27. The method of claim 26, wherein said modifying comprises
introducing a nucleic acid molecule comprising the human CXCL13 nucleic acid sequence into the genome of a rodent embryonic stem (ES) cell,
obtaining a rodent ES cell in which the human CXCL13 nucleic acid sequence has been integrated into an endogenous Cxcl13 locus to replace a rodent Cxcl13 genomic DNA thereby forming the humanized Cxcl13 gene, and
generating a rodent animal from the obtained rodent ES cell.

28. The method of claim 26 or 27, wherein the human CXCL13 nucleic acid sequence comprises exons 3-4 of a human CXCL13 gene.

29. The method of claim 26 or 27, wherein the human CXCL13 nucleic acid sequence encodes a polypeptide substantially identical with the mature protein sequence of the human CXCL13 protein.

30. The method of claim 29, wherein the human CXCL13 nucleic acid sequence comprises exon 3, exon 4 and the coding portion of exon 5 of a human CXCL13 gene.

31. The method of claim 29, wherein the human CXCL13 nucleic acid sequence comprises exons 3-5 of a human CXCL13 gene.

32. The method of claim 27, wherein the humanized Cxcl13 gene comprises exon 1 of the endogenous rodent Cxcl13 gene and exons 3-5 of a human CXCL13 gene, operably linked to the endogenous rodent Cxcl13 promoter at the endogenous rodent Cxcl13 locus.

33. The method of any one of claims 26-32, wherein the rodent is a mouse or a rat.

34. A targeting nucleic acid construct, comprising
a human CXCL13 nucleic acid sequence to be integrated into a rodent Cxcl13 gene at an endogenous rodent Cxcl13 locus, flanked by a 5′ nucleotide sequence and a 3′ nucleotide sequence that are homologous to nucleotide sequences at the rodent Cxcl13 locus,
wherein integration of the human CXCL13 nucleic acid sequence into the rodent Cxcl13 gene results in a replacement of a rodent Cxcl13 genomic DNA with the human CXCL13 nucleic acid sequence thereby forming a humanized Cxcl13 gene, and
wherein the human CXCL13 nucleic acid sequence encodes a polypeptide substantially identical with the chemokine IL-8 like domain of a human CXCL13 protein.

35. The targeting nucleic acid construct of claim 34, wherein the human CXCL13 nucleic acid sequence comprises exons 3-5 of a human CXCL13 gene.

36. The targeting nucleic acid of claim 34 or 35, wherein the rodent is a mouse or a rat.

37. A method of testing a candidate agent for treating a disease, comprising
introducing cells derived from a human subject suffering the disease into a genetically modified rodent animal as defined by any of claims 1-20,
contacting the rodent animal with a candidate agent, and
assaying if candidate agent is efficacious in reducing or eliminating the cells.

38. The method of claim 37, wherein the disease is cancer.

39. The method of claim 38, wherein the disease is a leukemia.

40. The method of claim 38 or 39, wherein the candidate agent is an anti-cancer compound, optionally selected from a small molecule compound, a nucleic acid molecule, or an antibody.

41. A rodent genome comprising a humanized Cxcl13 gene, wherein the humanized Cxcl13 gene comprises a rodent Cxcl13 nucleic acid sequence and a human CXCL13 nucleic acid sequence, and wherein the humanized Cxcl13 gene encodes a humanized Cxcl13 polypeptide comprising a chemokine IL-8 like domain substantially identical with the chemokine IL-8 like domain of a human CXCL13 protein.


SEQUENCE LISTING (29 sequences)

SEQ ID NO: 1 - DNA, 1219 bp, Homo sapiens
gagaagatgtttgaaaaaactgactctgctaatgagcctggactcagagctcaagtctgaactctacctccagacagaatgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctgatgctgatatttccactaagaacacctgcattcttcccttatccctgctctggattttagttttgtgcttagttaaatcttttccaggaaaaagaacttccccatacaaataagcatgagactatgtaaaaataaccttgcagaagctgatggggcaaactcaagcttcttcactcacagcaccctatatacacttggagtttgcattcttattcatcagggaggaaagtttctttgaaaatagttattcagttataagtaatacaggattattttgattatatacttgttgtttaatgtttaaaatttcttagaaaacaatggaatgagaatttaagcctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtggggggcggggccggggggactctggtatctaattctttaatgattcctataaatctaatgacattcaataaagttgagcaaacattttacttaaaaaaaaaaaaaaaaaa

SEQ ID NO: 2 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 3 - DNA, 1162 bp, Mus musculus
gagctaaaggttgaactccacctccaggcagaatgaggctcagcacagcaacgctgcttctcctcctggccagctgcctctctccaggccacggtattctggaagcccattacacaaacttaaaatgtaggtgttctggagtgatttcaactgttgtcggtctaaacatcatagatcggattcaagttacgccccctgggaatggctgccccaaaactgaagttgtgatctggaccaagatgaagaaagttatatgtgtgaatcctcgtgccaaatggttacaaagattattaagacatgtccaaagcaaaagtctgtcttcaactccccaagctccagtgagtaagagaagagctgcctgaagccactatcatctcaaaagacacacctgcacctttttttttatccctgctctgaattttagatatgttcttagttaaagaatttccaagaaaataactcccctctacaaacaaacatgactgtaggtaaaacaaagcaaaaacaaacaagcaaacaaacaaactaaaaaaaacccaatcctgcaggagctgagagggaatgctcaagctccgttgcatacccaacccacatccttgttccttaagaaaggctatttgagaacaggcatttagtgacaacccacttcagatgcatgtggtaatagatctgttgtttaatgttaaactatcctagattgtcgaggaatgaaaaacctacatgtcaaatgtgaacttgtagctcgtactaacaagaggtttgcgagatggacttcagttattttgcacccttgtaaaacgcaggcttccaaaatagtctccagaaggttcctgggaagctggtgcaatgccatcatgaggtggtgcaaagcaggtctcctttagagaaaagcttcctgggggaaacagtcctactttgaaaggttgcttgtataagatttattgtcttgcattaaaaccagtaacaattgaaagatcctcagcttaaaggtccaggctcttcagcagtatacaaatatattcctttgcactgtgaccctgatgatctatttttattattcatatctttcacacagacaaaataccagcctcttgtatcagattctttaatgtttcctattcatctggtgtcattcaataaatgtaatcaaatgttttgctta

SEQ ID NO: 4 - protein, 109 aa, Mus musculus
MRLSTATLLLLLASCLSPGHGILEAHYTNLKCRCSGVISTVVGLNIIDRIQVTPPGNGCPKTEVVIWTKMKKVICVNPRAKWLQRLLRHVQSKSLSSTPQAPVSKRRAA

SEQ ID NO: 5 - DNA, 1138 bp, Rattus norvegicus
gtgaactccacctccaggcagaatgaggctctgcaccgcagccctgcttcttctactggccatctgcctccctccaggccacggtattctggagacccattacacaaacttgaaatgtaggtgttccaaggtgagctcgacctttatcaatctaattctcgtagattggattcaagttatacgccctgggaatggctgccccaaaactgaaatcattttctggaccaaggccaagaaagctatatgtgtgaatcctactgccagatggttaccaaaagtattaaaatttgtccgaagaagtattacttcaactccccaagctccagtgagtaagaaaagagctgcctgaagccactgtcaccccaaaagacacctgcaccctttcttaatccctgctcggaatcttagtgttacgttcttagttgaagaatttccaagaaaataacttccctctacaaacacggctgtagattaaaagaaaaaaatcctgcagtagctgagaggagacactcgagctccttcccatactcaacccatattcttgttccttaagggaggatattttcgagcaggcatttagtgacaagccactttggtaatagacctgttgtttagtgttaaactatcctagaccctagaggaataaaagcatacatgtcgaatctgaacccatagctcctactaacaagaggtttatgagatggacttcagttagtttgcacccttgcaaaaatcaggcttccagaatagtttccagaaggtccctaagaagcagacgcattaccagcctaaggtgatgcagagcaggtctcctttagagagaatcttcatgagggaaataatgcttcgactttgaaaggttgcttgtacaaaatttattgtcttggattaaaaccagtaacactgaaagatcctcagcttaaagttccaggctccttaacagtatacaaatatattcctttgcactgtgaccctgctaatctatttttattattcacatctttcacacagacaaaataccagtctcttgtatcaaattctttaacgtttcctattcatccagtgtcattcaataaacttaatcaaataaaaaaaaaaaaaaaaaaaaaaaaaaaaaaaaaaaaaaaaaaaaaaaaaa

SEQ ID NO: 6 - protein, 108 aa, Rattus norvegicus
MRLCTAALLLLLAICLPPGHGILETHYTNLKCRCSKVSSTFINLILVDWIQVIRPGNGCPKTEIIFWTKAKKAICVNPTARWLPKVLKFVRRSITSTPQAPVSKKRAA

SEQ ID NO: 7 - DNA, 327 bp, Artificial Sequence
  CDS nucleic acid sequence encoding humanized, mouse/human chimeric Cxcl13 protein
atgaggctcagcacagcaacgctgcttctcctcctggccagctgcctctctccaggccacggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctga

SEQ ID NO: 8 - protein, 108 aa, Artificial Sequence
  humanized, mouse/human chimeric Cxcl13 protein
MRLSTATLLLLLASCLSPGHGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 9 - DNA, 22 bp, Artificial Sequence
  forward primer
ttacaggtgttctggaggtcta

SEQ ID NO: 10 - DNA, 23 bp, Artificial Sequence
  reverse primer
cgatcaatgaagcgtctagggat

SEQ ID NO: 11 - DNA, 28 bp, Artificial Sequence
  oligonucleotide probe
acacaagcttgaggtgtagatgtgtcca

SEQ ID NO: 12 - DNA, 21 bp, Artificial Sequence
  forward primer
gcctgaccaacatggagaaac

SEQ ID NO: 13 - DNA, 19 bp, Artificial Sequence
  reverse primer
gcctcagcctcccaagtag

SEQ ID NO: 14 - DNA, 25 bp, Artificial Sequence
  oligonucleotide probe
tacaaaattagccgggtgtggtggt

SEQ ID NO: 15 - DNA, 21 bp, Artificial Sequence
  forward primer
cctcgcagctttggattcaac

SEQ ID NO: 16 - DNA, 21 bp, Artificial Sequence
  reverse primer
gggcagtttgggatctggaat

SEQ ID NO: 17 - DNA, 29 bp, Artificial Sequence
  oligonucleotide probe
tgttccacaaatggttgttctcttattcc

SEQ ID NO: 18 - DNA, 24 bp, Artificial Sequence
  forward primer
ggcatcaaactcagagatgtgaag

SEQ ID NO: 19 - DNA, 21 bp, Artificial Sequence
  reverse primer
gacaggcaagagaccagtcat

SEQ ID NO: 20 - DNA, 27 bp, Artificial Sequence
  oligonucleotide probe
tctagccatgacctacttgggagtagt

SEQ ID NO: 21 - DNA, 24 bp, Artificial Sequence
  forward primer
tcggttctacgtctatgttctttg

SEQ ID NO: 22 - DNA, 23 bp, Artificial Sequence
  reverse primer
gggcttccagaatacctgtgaac

SEQ ID NO: 23 - DNA, 25 bp, Artificial Sequence
  probe primer
tccaatgggttgaagtgtctgactc

SEQ ID NO: 24 - DNA, 20 bp, Artificial Sequence
  forward primer
tgcacagcagccatcatttg

SEQ ID NO: 25 - DNA, 22 bp, Artificial Sequence
  reverse primer
caggcagctcttctcttactca

SEQ ID NO: 26 - DNA, 27 bp, Artificial Sequence
  oligonucleotide probe
tgccactagaggaaagctatggtttcc

SEQ ID NO: 27 - DNA, 17 bp, Artificial Sequence
  forward primer
tgcggccgatcttagcc

SEQ ID NO: 28 - DNA, 18 bp, Artificial Sequence
  reverse primer
ttgaccgattccttgcgg

SEQ ID NO: 29 - DNA, 21 bp, Artificial Sequence
  oligonucleotide probe
acgagcgggttcggcccattc